(19) United States
(12) Patent Application Publication
(10) Pub. No.: US 2026/0007403 A1
(43) Pub. Date: Jan. 8, 2026

(54) SURGICAL TOOL WITH TENSION TRACKING SYSTEM

(71) Applicant: The Government of the United States, as Represented by the Director of the Defense Health Agency, Fort.Detrick, MD (US)

(72) Inventor: Daniel Joseph Cognetti, San Antonio, TX (US)

(21) Appl. No.: 19/245,957

(22) Filed: Jun. 23, 2025

Related U.S. Application Data
(60) Provisional application No. 63/667,863, filed on Jul. 5, 2024.

(51) Int. Cl. A61B 17/062; A61B 17/00; A61B 17/04; A61B 90/00

(52) U.S. Cl. CPC A61B 17/062; A61B 2017/00128; A61B 2017/0464; A61B 2017/0496; A61B 2090/064

(57) ABSTRACT

A tension tracking system for a surgical tool includes a spring embedded within the tool and tension markings aligned to indicate displacement of the spring, wherein application of tension during use of the tool causes displacement in the spring which can be visually seen and indicted by the tension markings.

### RELATED APPLICATIONS

This application claims the benefit of U.S. Provisional Application No. 63/667,863, filed Jul. 5, 2024, the entire contents of which is incorporated herein by reference in its entirety.

### GOVERNMENT INTEREST

The disclosure described herein may be manufactured, used, and licensed by or for the U.S. Government.

### FIELD

The present disclosure relates generally to surgical tools. More particularly, the present disclosure relates to a surgical tool with a tension-detecting mechanism.

### BACKGROUND

Undue pressure or tension across a wound after suture closure can result in tissue necrosis and ischemia. As such, an improved system which assists surgeons in monitoring tension across a wound closure is needed.

### SUMMARY

In some forms, there is a surgical tool that includes a tension tracking system, which can measure a tensile force applied by a clinician.

In some forms, disclosed is a tension tracking system for a surgical tool, which includes at least one spring embedded within the tool and tension markings aligned to indicate displacement of the spring, wherein application of tension during use of the tool causes displacement in the spring which can be visually seen and indicted by the tension markings.

In some forms, a surgical tool for performing sutures includes a first section, a second section, a first tension sensor, and a second tension sensor. The first section has a first end with a first gripping portion and a second end with a first jaw. The second section is pivotably connected to the first section. The second section includes a third end with a second gripping portion and a fourth end with a second jaw. The first tension sensor is disposed between the first end and the second end. The second tension sensor is arranged in parallel with the first tension sensor and disposed between the third end and the fourth end. The first section and the second section can move between a closed position where the first jaw contacts the second jaw and an open position where the first jaw is spaced apart from the second jaw. The first jaw and the second jaw can grasp a suture in the closed position. In the closed position, the first tension sensor and the second tension sensor can measure a tensile force applied at the first gripping portion and the second gripping portion.

In some forms, a surgical tool for performing sutures includes a first section, a second section, and a first tension sensor. The first section has a first end with a first gripping portion and a second end with a first jaw. The second section is pivotably connected to the first section. The second section includes a third end with a second gripping portion and a fourth end with a second jaw. The first tension sensor is disposed between the first end and the second end. The first tension sensor is a coil spring. The first section and the second section can move between a closed position where the first jaw contacts the second jaw and an open position where the first jaw is spaced apart from the second jaw. The first jaw and the second jaw can grasp a suture in the closed position. In the closed position, the first tension sensor can measure a tensile force applied at the first gripping portion and the second gripping portion.

In some forms, a method of applying a suture to a patient includes determining a tension threshold and grasping a string for use in the suture with a surgical tool. The surgical tool includes a first tension sensor and a second tension sensor arranged in parallel with the first tension sensor. The method also includes applying tension to the string via the surgical tool. The method further includes monitoring the applied tension via a first output from the first tension sensor and a second output from the second tension sensor. Finally, the method includes comparing the first output and the second output to the tension threshold and adjusting the applied tension based on the comparison.

The disclosure herein should become evident to a person of ordinary skill in the art given the following enabling description and drawings. The drawings are for illustration purposes only and are not drawn to scale unless otherwise indicated. The drawings are not intended to limit the scope of the disclosure. The following enabling disclosure is directed to one of ordinary skill in the art and presupposes that those aspects within the ability of the ordinarily skilled artisan are understood and appreciated.

### BRIEF DESCRIPTION OF THE FIGURES

The detailed description of some examples of the disclosure will be made below with reference to the accompanying figures, wherein the figures disclose one or more examples of the present disclosure.

FIG. 1 is a perspective view of a surgical tool with a tension tracking system according to certain examples.

FIG. 2 is a top view of the surgical tool of FIG. 1.

FIG. 3 is a detailed view of the surgical tool of FIG. 1, depicting motion in the tension tracking system

### DETAILED DESCRIPTION OF CERTAIN EXAMPLES

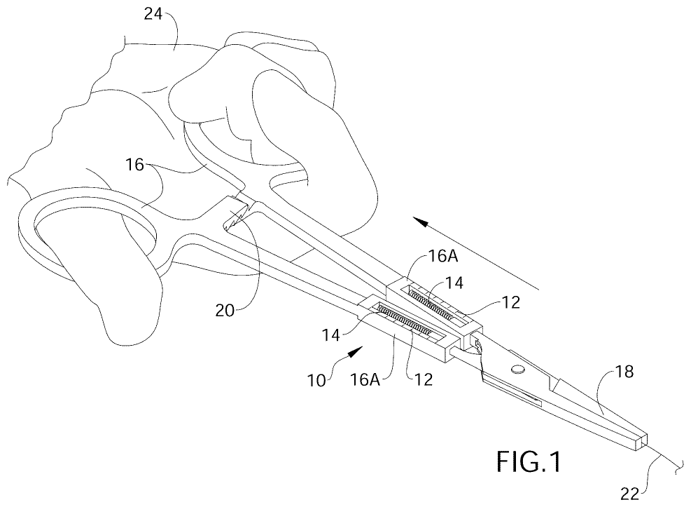

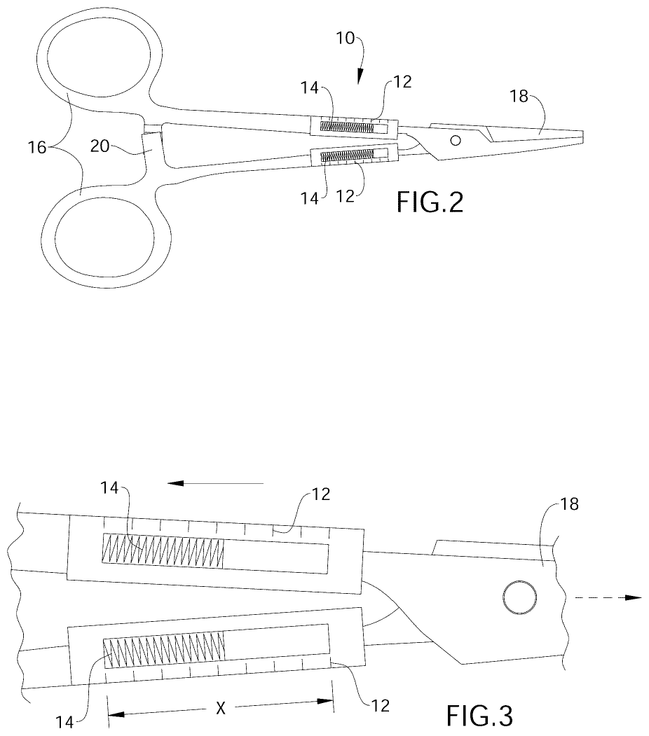

According to various examples, as depicted in FIGS. 1-3, disclosed is a surgical tool 20 (e.g., a needle driver) which incorporates a tension tracking system 10 that can indicate tension during use of the tool 20. As described in more detail below, the tension tracking system 10 can assist a clinician determine a tension that they are applying during a procedure (e.g., during a suture) to reduce or eliminate over-tensioning.

In some forms, the tool 20 may be used to hold a suturing needle or strings during surgical procedures and may generally comprise a pair of hinged jaws 18 wherein each jaw 18 is coupled to a finger grip 16, as depicted.

In certain examples, the tension tracking system 10 may be included on the tool 20 between the finger grips 16 and the hinged jaws 18. The tension tracking system 10 may provide a tension gauge assembly that can measure the tension applied to a suturing needle or strings (e.g., when a clinician provides a pulling force via the finger grips 16). In the illustrated example, the tension tracking system 10 may include a pair of biasing members 14 (e.g., coil springs), with one biasing member 14 coupled between each jaw 18 and its respective finger grip 16. The biasing members 14 may be arranged in parallel and may be disposed approximately the same distance between the respective finger grip 16 and jaw 18. Other examples may have a different arrangement of biasing members 14, like a different number of biasing members 14 and/or one or more biasing members located in a different position.

In some forms, each finger grip 16 includes a stem that incorporates a window block 16A for holding spring 14. An end of each jaw opposite the grip end, may be engaged through a bottom opening in a respective window block 16A and coupled to a respective biasing member 14, wherein each biasing member is positioned in alignment between each jaw 18 and respective finger grip 16 to ensure force transmission to the biasing members 14.

In some forms, each window block 16A may be at least partially transparent or translucent to permit a clinician to view the biasing members 14 during use of the tool 20. For example, the clinician may observe the distance that the biasing members 14 have compressed to determine a tension being applied by the tool 20. In other examples, each biasing member 14 may not be covered within the window block 16A.

In certain forms, markings 12 may be disposed on a surface of the tool 20. For example, the markings 12 may be disposed on a surface of the window block 16A. The illustrated markings 12 may be a series of lines indicating a distance (e.g., a ruler). In other examples, the markings 12 may be different colors (e.g., green, yellow, red). In still other examples, the markings 12 may be alphanumeric characters.

In other examples (not shown), one or more of the biasing members 14 may be replaced with electronic sensors (e.g., a resistance sensor). The electronic sensor may similarly measure the forced applied by a clinician onto the tool 20.

During a suturing procedure, the gripping end of jaws 18 may be used to hold or clamp strings 22 (or a surgical needle). The biasing members 14 may begin in a neutral position, which may extend along the length of the respective window block 16A. A surgeon 24 holding needle driver 20 at finger grips 16 may pull on strings 22 thereby applying tension to the string 22. This causes a displacement (e.g., compression) in the biasing members 14, which can be seen within window block 16A and is indicated by tension markings 12. As such, the surgeon may have a visual indication of the degree of tension being applied on strings 22. In some forms, the markings 12 may indicate a distance of compression of the biasing members 14. In other examples, the markings 12 may generally indicate an acceptable applied force (e.g., the biasing member compressing into a green zone), a slightly overapplied force (e.g., the biasing member 14 compressing into a yellow zone), and an overapplied force (e.g., the biasing member 14 compressing into a red zone). It shall be appreciated that in alternate examples, tension tracking system 10 may be used to indicate a push or compression force (e.g., on a needle during surgical procedure).

Thus, the disclosed subject matter provides a modified handheld surgical instrument designed to grasp and manipulate needles and sutures with an embedded tension meter that indicates a force exerted across a wound in real time. As such, the surgeon need not only rely on subjective tactile feel. In examples, the surgeon may grip the handle and manipulate the needle driver to open and close the jaws which allow the surgeon to pull on the suture to tie and secure a knot for wound closure. While pulling on the suture with jaws, the tension sensors within the device measure the force exerted on the tissue. This data is displayed in real-time, allowing the surgeon to monitor tension levels.

In some forms, a threshold may be determined for a given procedure, which may indicate a maximum tensile force (or range of tensile forces) that may be applied to the string 22 to avoid injury to the patient. The threshold may vary depending on the specific procedure. The clinician may calibrate the tension tracking system 10 based on a specific threshold. For example, the clinician may move place an indicator along the marking 12 to illustrate the threshold and/or change the size of the colored zones. In electronic examples, the tension tracking system 10 may include a display and a processor, where the threshold can be programmed for a specific procedure.

In certain forms, the tension tracking system 10 may include a feedback mechanism, which may provide an indication (e.g., other than the visual indication of the markings 12) to the clinician that the threshold is exceeded. In some examples, the feedback mechanism may include tactile feedback, which may alert the clinician that a threshold has been exceeded. The tension tracking system 10 can include different patterns of tactile responses to communicate different information (e.g., a different tactile response for the clinician approaching the threshold than for when the clinician exceeds the threshold). The tactile response may be produced mechanically (e.g., because of the compression of the biasing member 14) or from an electronic mechanism. In some examples, the feedback mechanism may include an auditory response which may alert the clinician that a threshold has been exceeded. The auditory response may produce one or more sounds to similarly communicate information to the clinician as the threshold is reached and/or exceeded. The tension tracking system 10 may include the tactile and/or auditory alert, and/or any other similar feedback mechanism.

In some alternate examples, the disclosed system may incorporate an analog or digital display. In further alternate examples, tension data may be transmitted (e.g., wirelessly) and recorded, which may aid in diagnostics, training, and/or research purposes. For example, data may be transmitted to a remote computer, which may process the data and provide feedback for the clinician (e.g., indications and/or alerts that a threshold was exceeded).

The disclosed system assists surgeons in adjusting their technique to achieve optimal tension distribution, for proper wound healing and minimized risk of complications. As the system provides objective feedback on wound tension and the risk of wound complications, it may lead to more beneficial treatment decisions, such as closing a wound or leaving a wound partially or completely open, based on individualized patient needs. The disclosed system may also provide a means for further research to assess the contribution of wound tension on intramuscular and compartment pressure in compartment syndrome.

In one example, the disclosed tool may be made of stainless steel, and produced in accordance with medical grade equipment standards. It shall be appreciated that tools manufactured in accordance with the disclosed system may comprise any alternative known materials in the field and may be manufactured and assembled using any known techniques in the field. It shall be appreciated that the disclosed system may be applied to other tools in the surgical field, and/or to tools used in other application in alternate examples. It shall be appreciated that the disclosed device and system can have multiple configurations in different examples. It shall be appreciated that the device and system described herein may be of any size, and/or dimensions.

It shall be understood that the orientation or positional relationship indicated by terms such as “upper”, “lower”, “front”, “rear”, “left”, “right”, “top”, “bottom”, “inside”, “outside” is based on the orientation or positional relationship shown in the accompanying drawings, which is only for convenience and simplification of describing the disclosed subject matter, rather than indicating or implying that the indicated device or element must have a specific orientation or are constructed and operated in a specific orientation, and therefore should not be construed as a limitation of the present invention.

As used herein, the articles “a” and “an” are intended to include one or more items, and may be used interchangeably with “one or more.” Where only one item is intended, the term “one” or similar language is used. Also, as used herein, the terms “has”, “have”, “having”, “with” or the like are intended to be open-ended terms. Further, the phrase “based on” is intended to mean “based, at least in part, on” unless explicitly stated otherwise.

The constituent elements of the disclosed device and system listed herein are intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device. Terms such as ‘approximate,’ ‘approximately,’ ‘about,’ etc., as used herein indicate a deviation of within +/−10%. Relationships between the various elements of the disclosed device as described herein are presented as illustrative examples only, and not intended to limit the scope or nature of the relationships between the various elements. Persons of ordinary skill in the art may appreciate that numerous design configurations may be possible to enjoy the functional benefits of the inventive systems. Thus, given the wide variety of configurations and arrangements of examples of the present invention the scope of the invention is reflected by the breadth of the claims below rather than narrowed by the examples described above.

One of ordinary skill will appreciate that the exact dimensions and materials are not critical to the disclosure and all suitable variations should be deemed to be within the scope of the disclosure if deemed suitable for carrying out the objects of the disclosure.

One of ordinary skill in the art will also readily appreciate that it is well within the ability of the ordinarily skilled artisan to modify one or more of the constituent parts for carrying out the various examples of the disclosure. Once armed with the present specification, routine experimentation is all that is needed to determine adjustments and modifications that will carry out the present disclosure.

One of ordinary skill in the art will understand that the words “surgeon”, “clinician”, “technician”, or “user” may be used interchangeably to refer to any person who may use the disclosed subject matter.

The above examples are for illustrative purposes and are not intended to limit the scope of the disclosure or the adaptation of the features described herein. Those skilled in the art will also appreciate that various adaptations and modifications of the above-described preferred examples can be configured without departing from the scope and spirit of the disclosure. Therefore, it is to be understood that, within the scope of the appended claims, the disclosure may be practiced other than as specifically described.

What is claimed is:

1. A surgical tool for performing sutures, the surgical tool comprising:
a first section having a first end with a first gripping portion and a second end with a first jaw;
a second section pivotably connected to the first section, wherein the second section includes a third end with a second gripping portion and a fourth end with a second jaw;
a first tension sensor disposed between the first end and the second end; and
a second tension sensor arranged in parallel with the first tension sensor and disposed between the third end and the fourth end;
wherein the first section and the second section are configured to move between a closed position where the first jaw contacts the second jaw and an open position where the first jaw is spaced apart from the second jaw;
wherein the first jaw and the second jaw are configured to grasp a suture in the closed position; and
wherein in the closed position, the first tension sensor and the second tension sensor are configured to measure a tensile force applied at the first gripping portion and the second gripping portion.

2. The surgical tool of claim 1, wherein the first tension sensor is a first coil spring and the second tension sensor is a second coil spring.

3. The surgical tool of claim 2, wherein the first coil spring is movable between a first position and a second position, wherein the first position is a neutral position when no force is applied at the first gripping portion and the second gripping portion, and wherein the second position is a compressed position when a force is applied at the first gripping portion and the second gripping portion.

4. The surgical tool of claim 2, wherein the first section further includes a first window block and the second section further includes a second window block, wherein the first window block at least partially surrounds the first coil spring and includes a first transparent portion to permit observation of the first coil spring, and wherein the second window block at least partially surrounds the second coil spring and includes a second transparent portion to permit observation of the second coil spring.

5. The surgical tool of claim 1, wherein the first section includes first markings proximate to the first tension sensor and the second section includes second markings proximate to the second tension sensor, wherein the first markings are configured to communicate a first output from the first tension sensor, and wherein the second markings are configured to communicate a second output from the second tension sensor.

6. The surgical tool of claim 5, wherein the first markings are adjustable by a user to correspond to a predetermined maximum allowable tension.

7. The surgical tool of claim 1, wherein the first tension sensor and the second tension sensor are electronic sensors.

8. A surgical tool for performing sutures, the surgical tool comprising:
a first section having a first end with a first gripping portion and a second end with a first jaw;
a second section pivotably connected to the first section, wherein the second section includes a third end with a second gripping portion and a fourth end with a second jaw;
a first tension sensor disposed between the first end and the second end, wherein the first tension sensor is a coil spring;
wherein the first section and the second section are configured to move between a closed position where the first jaw contacts the second jaw and an open position where the first jaw is spaced apart from the second jaw;
wherein the first jaw and the second jaw are configured to grasp a suture in the closed position; and
wherein in the closed position, the first tension sensor is configured to measure a tensile force applied at the first gripping portion and the second gripping portion.

9. The surgical tool of claim 8, wherein the first coil spring is movable between a first position and a second position, wherein the first position is a neutral position when no force is applied at the first gripping portion and the second gripping portion, and wherein the second position is a compressed position when a force is applied at the first gripping portion and the second gripping portion.

10. The surgical tool of claim 8, wherein the first section further includes a first window block, and wherein the first window block at least partially surrounds the first coil spring and includes a first transparent portion to permit observation of the first coil spring.

11. The surgical tool of claim 8, wherein the first section includes first markings proximate to the first tension sensor, and wherein the first markings are configured to communicate a first output from the first tension sensor.

12. The surgical tool of claim 11, wherein the first markings are adjustable by a user to correspond to a predetermined maximum allowable tension.

13. The surgical tool of claim 8, wherein the first tension sensor is an electronic sensor.

14. A method of applying a suture to a patient, wherein the method comprises:
determining a tension threshold;
grasping a string for use in the suture with a surgical tool, wherein the surgical tool includes a first tension sensor and a second tension sensor arranged in parallel with the first tension sensor;
applying tension to the string via the surgical tool;
monitoring the applied tension via a first output from the first tension sensor and a second output from the second tension sensor;
comparing the first output and the second output to the tension threshold; and
adjusting the applied tension based on the comparison.

15. The method of claim 14, wherein the first tension sensor is a first coil spring and the second tension sensor is a second coil spring.

16. The method of claim 15, further comprising moving the first coil spring between a first position and a second position, wherein the first position is a neutral position when no force is applied, and wherein the second position is a compressed position when a force is applied.

17. The method of claim 15, wherein the surgical tool further includes a first window block and a second window block, wherein the first window block at least partially surrounds the first coil spring and includes a first transparent portion to permit monitoring of the first coil spring, and wherein the second window block at least partially surrounds the second coil spring and includes a second transparent portion to permit monitoring of the second coil spring.

18. The method of claim 15, wherein monitoring further includes determining the first output by observing a position of the first coil spring in relation to a first marking on the surgical tool, and determining the second output by observing a position of the second coil spring in relation to a second marking on the surgical tool.

19. The method of claim 14, wherein the first tension sensor and the second tension sensor are electronic sensors wirelessly connected to a computer, the method further comprising,
programming the tension threshold into the computer;
communicating the first output and the second output to the computer; and
providing a third output from the computer configured to communicate how the applied tension should be adjusted.

20. The method of claim 14, further comprising outputting an auditory and/or tactile response where the first output and/or the second output exceed the tension threshold.
